(19) United States
(12) Patent Application Publication
(10) Pub. No.: US 2021/0244316 A1
(43) Pub. Date: Aug. 12, 2021

(54) SMART WATCH FOR TREMOR MONITORING AND CONTROL

(71) Applicant: Prince Mohammad Bin Fahd University, Dhahran (SA)

(72) Inventor: Nassim KHALED, Dhahran (SA)

(73) Assignee: Prince Mohammad Bin Fahd University, Dhahran (SA)

(21) Appl. No.: 16/787,790

(22) Filed: Feb. 11, 2020

(51) Int. Cl. A61B 5/11; A61H 23/00

(52) U.S. Cl. CPC A61B 5/1101; A61H 23/00; A61H 2230/065; A61B 5/681; A61H 2201/5092; A61H 2230/505; A61H 2201/165; A61H 2230/208

(57) ABSTRACT

A smart watch for tremor monitoring and control of tremors in an extremity of a user, comprises a wearable band including a camera, a plurality of motors, an onboard communications module configured to communicate with a cloud server storing a tremor control application, and a computing system including circuitry and a processor having program instructions configured to actuate the camera to take a series of images, generate an analysis of the series of images and actuate the motors to generate tremor control vibrations based on the analysis of the series of images. The tremor control application stores a body profile and performs a high level analysis of the tremor reduction effect. Motor tuning parameters are communicated to the smart watch to improve the tremor reduction effect.

### BACKGROUND

### Technical Field

The present disclosure is directed to a smart watch for tremor monitoring and control, and a method of its use. The smart watch detects tremors from camera images of the surrounding environment and actuates vibrators to offset the tremors.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

A tremor is an involuntary muscle contraction leading to shaking or cyclic movement in one or more parts of the body. The muscle contraction often follows a rhythmic pattern. It is common for tremor to affect the hand or wrist area of a sufferer. Tremor may be intermittent or in some cases may be constant or permanent. In some cases, tremor accompanies or is accompanied by one or more other disorders. The effects of tremor can be partially or severely disabling, and are often the cause of embarrassment. Some forms of tremor include essential tremor, restless leg syndrome (RLS), Parkinson's tremor, dystonic tremor, cerebellar tremor, resting tremor, action tremor, psychogenic tremor (related to psychological disorders), enhanced physiologic tremor, or orthostatic tremor, (See: WO2019046180A1, incorporated herein by reference in its entirety).

Tremor at rest (also known as rest tremor or resting tremor) is an easily (visually) recognized motor symptom of Parkinson's disease. It typically manifests itself as an involuntary, unilateral (onesided) shaking of an extremity (e.g. hand, foot, etc.). Extremities of the upper body, such as the hands, are more affected than those of the lower body. The shaking occurs at a frequency between 4-12 Hz. This type of tremor is only present when muscles are at rest, hence the name “tremor at rest”, and is not present during sleep or during voluntary movement of the extremity. The effectiveness of therapeutic interventions (e.g. medication) can be partially measured through tremor assessment in Parkinson's disease patients. Small, wearable sensor devices have been used for tremor assessment. (See: Ahlrichs, C; Sama, A, “Is “Frequency Distribution” Enough to Detect Tremor in PD Patients Using a Wrist Worn Accelerometer? PervasiveHealth, May 20-23, 2014, Oldenburg, Germany, incorporated herein by reference in its entirety).

U.S. Pat. No. 9,452,287B2 describes treating tremors by sending an electrical, mechanical or chemical stimulus through a peripheral nerve site of the hands. The stimulation normalizes the neural firing in the nerve and reduces the tremor. Peripheral nerve stimulation is believed to treat tremors by dephasing, overriding or obscuring abnormal brain circuit dynamics.

The application of external mechanical noise has been shown to reduce tremors, by a process known as “stochastic resonance”. In a study, a mechanical stimulation as well as white noise was applied to the hands of a group of patients. All patients reported that the noise stimulation with mechanical stimulation reduced the tremors more than mechanical stimulation alone. (See: Trenado, C, Amtage, F, Kristeva, R., “Suppression of Enhanced Physiological Tremor via Stochastic Noise: Initial Observations”, Nov. 14, 2014, PLoS One. 2014; 9(11): e112782, doi: 10.1371/journal.pone.0112782, incorporated herein by reference in its entirety).

The references above describe various ways to monitor and treat tremors. However, none describes a single device which detects tremors by monitoring the environment with cameras.

Accordingly, it is one object of the present disclosure to provide methods and systems for a smart watch which monitors tremors by detecting changes in the surrounding environment and treats the tremors by noise and mechanical stimulation of the nerves in the wrists.

### SUMMARY

The first embodiment describes a smart watch for tremor monitoring and control of an extremity of a user, comprising a wearable band including a camera, a plurality of motors, an onboard communications module configured to communicate with a cloud server storing a tremor control application, and a computing system including circuitry and a processor having program instructions configured to actuate the camera to take a series of images, generate an analysis of the series of images and actuate the motors to generate tremor control vibrations based on the analysis of the series of images.

In another exemplary embodiment, a method for monitoring and controlling tremors in an extremity of a patient by a smart watch comprises actuating a camera located on the extremity to acquire a first series of images of an environment surrounding the extremity, analyzing the first series of images to determine a magnitude and frequency of a tremor, actuating at least one of a plurality of motors located on the extremity to generate tremor control vibrations based on the analysis of the first series of images. The analysis of the first series of images includes subtracting each image from an image having an immediately previous timestamp to generate a time series of differences, normalizing the differences, comparing a magnitude of each difference to a threshold, comparing a magnitude of each difference to a threshold, identifying a tremor occurrence if the magnitude is greater than the threshold, and determining a frequency of the tremors by mapping the tremor occurrences with respect to the timestamps. The method further comprises retrieving log in credentials from a memory of the smart watch, transmitting the log in credentials, magnitudes and frequencies in a data packet to a tremor control application, receiving the data packet by the tremor control application, matching the log in credentials to a database of access codes of the tremor control application, retrieving a body profile of the patient from the database, generating, by the tremor control application, motor tuning parameters based on the magnitudes, frequencies and the body profile, transmitting the motor parameters to the smart watch, receiving, by the smart watch, the motor parameters, and generating commands by pulse width modulation to actuate each motor according to the motor tuning parameters. The method includes monitoring the tremor reduction effect of the vibrations and storing the tremor reduction effect in the body profile for use in generating the motor tuning parameters.

In another exemplary embodiment, a smart watch system for monitoring and controlling tremors in an extremity of a patient is described, comprising a wearable band surrounding the extremity, the wearable band including a camera configured to acquire a first series of images of an environment surrounding the extremity, an image processor configured to receive the first set of images and timestamp each image, an image comparator configured to subtract each image from an image having an immediately previous timestamp to generate a time series of differences, a tremor analysis module configured normalize the differences, compare the magnitude of each difference to a threshold, identify a tremor occurrence if the magnitude is greater than the threshold and determine a frequency of the tremors by mapping the tremor occurrences with respect to the timestamps, a plurality of sensors including at least one of a body temperature sensor, an O2 pulse oximeter and a heart rate monitor the plurality of sensors configured to take measurements of the extremity, a plurality of motors configured to generate at least one of vibrations in the extremity and vibrations in the extremity and audible frequencies, a pulse width modulator configured to generate commands to actuate the motors to generate the vibrations, a memory storing log in credentials, a computing system including circuitry and a processor having program instructions configured to actuate the camera to acquire the first series of images, instruct the tremor analysis module to determine the frequency and magnitude of the tremors, actuate at least one of the plurality motors to generate tremor control vibrations based on the analysis of the series of images, monitor a first tremor reduction effect due to the vibration of the at least one motor, generate a data packet including the at least one sensor measurement, the magnitude and frequency of the tremor occurrence, the first tremor reduction effect and the log in credentials, an onboard communications module configured to transmit the data packet to a cloud server, a tremor control application stored on the cloud server, the tremor control application including a transceiver configured to receive the data packets, a registration module configured to register a computing device of the patient with the tremor control application and to generate a unique access code, an application memory configured to store unique access codes of a plurality of smart watch patients registered with the tremor control application, a database configured to store a body profile of the patient, application circuitry and an application processor having program instructions configured to match the log in credentials to one of the unique access codes, retrieve the body profile of the user associated with the unique access code, update the database the at least one sensor measurement, the magnitude and frequency of the tremor occurrence, the first tremor reduction effect, compare the at least one sensor measurement, the magnitude and frequency of the tremor occurrence, the first tremor reduction effect to the body profile, generate motor tuning to reduce the tremors, wherein the motor tuning parameters include at least an amplitude and frequency for each motor of the smart watch, send the motor tuning parameters to the transceiver, wherein the transceiver is further configured to transmit the motor tuning parameters to the onboard communications module of the smart watch.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is an overview of the smart watch system, according to certain embodiments.

FIG. 2A illustrates the positioning of the motors on the wearable band on the front of a hand or wrist, according to certain embodiments.

FIG. 2B illustrates the positioning of the motors on the wearable band on the back of a hand or wrist, according to certain embodiments.

FIG. 2C is an image of the vibratory motors.

FIG. 2D illustrates the wearable band and the placement of the camera, motors and sensors.

FIG. 3 is a block diagram of the computing system of the smart watch, according to certain embodiments.

FIG. 4A, 4B are an exemplary flowchart of the motor actuation and monitoring, according to certain embodiments.

FIG. 5A is a graph of the differences in the series of camera images plotted against time, according to certain embodiments.

FIG. 5B represents the probability distribution the PWM command of each motor, according to certain embodiments.

FIG. 6 is a block diagram of the computing environment of the tremor control application, according to certain embodiments.

FIG. 7 is an illustration of a non-limiting example of details of computing hardware used in the computing system, according to certain embodiments.

FIG. 8 is an exemplary schematic diagram of a data processing system used within the computing system, according to certain embodiments.

FIG. 9 is an exemplary schematic diagram of a processor used with the computing system, according to certain embodiments.

FIG. 10 is an illustration of a non-limiting example of distributed components which may share processing with the controller, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise. The drawings are generally drawn to scale unless specified otherwise or illustrating schematic structures or flowcharts.

Furthermore, the terms “approximately,” “approximate,” “about,” and similar terms generally refer to ranges that include the identified value within a margin of 20%, 10%, or preferably 5%, and any values therebetween.

Tremors represent a family of diseases, and include tremors such as essential tremor, restless leg syndrome (RLS), Parkinson's tremor, dystonic tremor, cerebellar tremor, resting tremor, action tremor, psychogenic tremor (related to psychological disorders), enhanced physiologic tremor, or orthostatic tremor. These tremors affect the upper limbs, especially the hands, and interfere with basic activities of daily living, such as handwriting, dressing, eating and self-care.

Essential tremor shows a frequency of between 4-12 Hz and is of variable amplitude. The frequency is higher than that of normal, voluntary, human movements, thus tremors can be detected on the basis of the frequency difference.

Aspects of the present disclosure are directed to a smart watch for tremor monitoring and control of tremors in an extremity of a user, a method for monitoring and controlling tremors in an extremity of a patient by a smart watch, and a smart watch system for monitoring and controlling tremors in an extremity of a patient.

A smart watch may refer to a wearable technology worn on the wrist, hand, leg, or finger (or any other body extremity) that contains one or more computing units with a CPU and memory and one or more communication units such as but not limited to Wi-Fi, Bluetooth, or cellular modem units (3G, 4G, LTE) capable of relaying information wirelessly. A smart watch may contain mobile software or a mobile application to provide a user interface that allows the user to enter log in credentials, view current and past information graphically or numerically, and provides access to data from onboard sensors, should they exist. Information may be sent from the smart watch which may include login credentials as well as motion data from onboard sensors, to a remote server computing cloud platform for data analysis.

In aspects of the present disclosure, a smart watch identifies tremors by detecting changes in camera images of the surrounding environment. The smart watch includes actuators, such as vibrating motors and audio, that are used to reduce tremors. The vibrating motors vibrate the nerves in the wrist, which acts as “white noise” feedback as perceived by the brain. Some or all of the vibrating motors may also produce audible noise to aid in reducing the tremors.

The smart watch of the present disclosure is a wearable device. At least one camera is used with image processing to detect and quantify tremors.

In an aspect, the smart watch may include sensors which record O₂, heart rate and body temperature. These readings may be used to provide a health report to the patient.

Acquired image and sensor data is processed onboard the smart watch, and then transmitted to a tremor control computer application in a cloud computing platform when connectivity is available. The computer application stores a tremor model of the user, which is updated based on the new data. The images are analyzed to determine whether there is an onset of tremor. When tremors are detected, the computer application determines the frequency of the tremors from the camera images and generates tuning parameters, which are sent to the onboard device of the smart watch. The onboard device provides commands to the audio and vibration actuators based on the tuning parameters.

In an aspect of the present disclosure, a smart watch is worn on a patient's wrist. Each smart watch includes a camera which takes images of the surrounding environment at periodic intervals. Subsequent images are subtracted and the severity of the tremor is determined by the magnitude of the difference. Based on the magnitude, the range of frequencies and amplitudes for actuating motors in the respective smart watch are determined. The motors may each be actuated by a different range of frequencies or a different vibration amplitude, and the motors on the different parts of the wrist are not necessarily actuated symmetrically.

In an aspect, each motor may have a different operating frequency range and provide different amplitudes of mechanical or audio stimulation.

In another aspect, some motors are adapted to generate acoustic sounds and some are adapted to generate mechanical vibration.

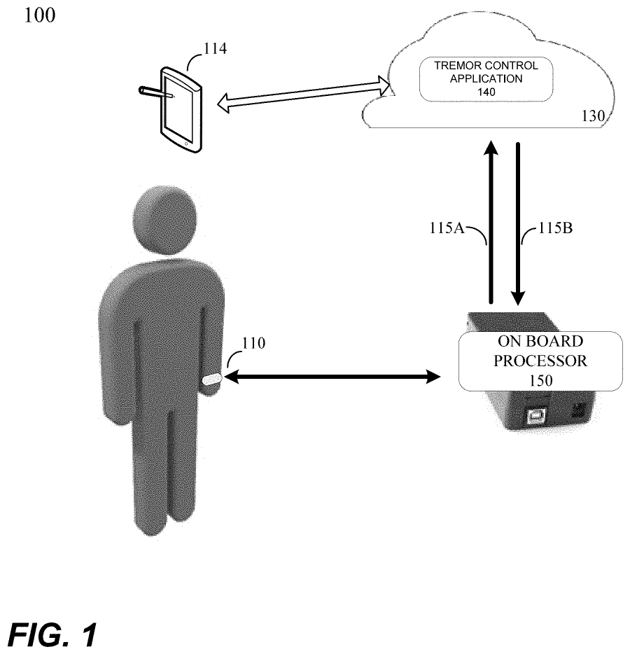

FIG. 1 shows an overview of the smart watch system. Smart watch 110 is worn on the wrist of the patient to control the tremors of the respective hand. The smart watch includes an image processor and an onboard communications module. The onboard communications module may be configured to transmit and receive data over LTE, WiFi and Bluetooth. The smart watch further includes a camera configured to take successive images of the surrounding environment. An onboard processor 150 within the smart watch is configured to receive the successive images, subtract the images and detect a tremor occurrence from the differences between the successive images. The tremor occurrences, magnitudes and frequencies are transmitted by the onboard communications module (see transmission arrow 115A) to a tremor control application 140 stored in a cloud computing platform 130.

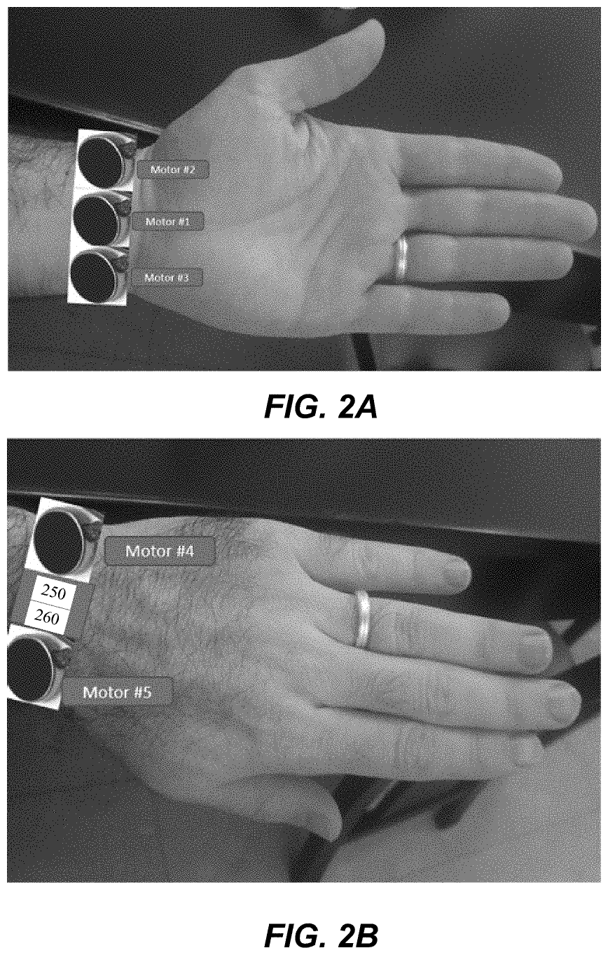

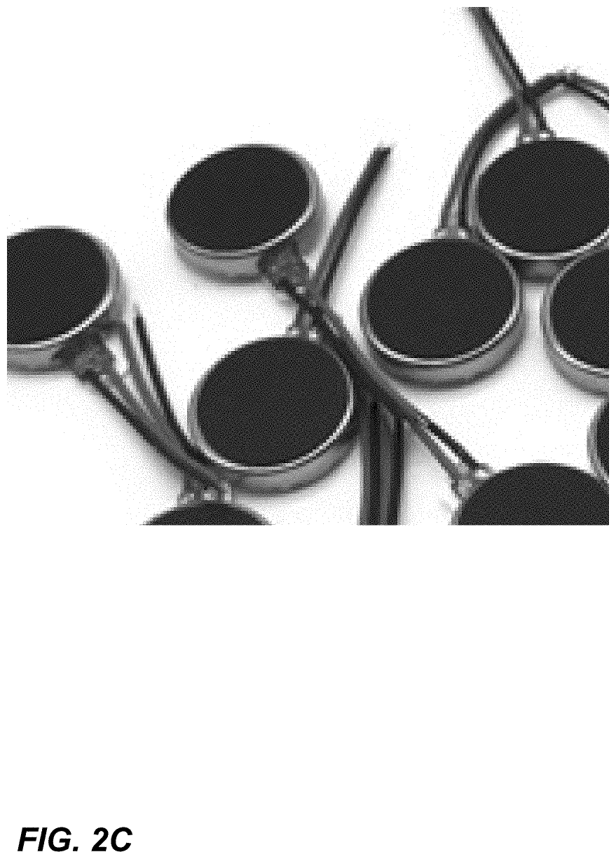

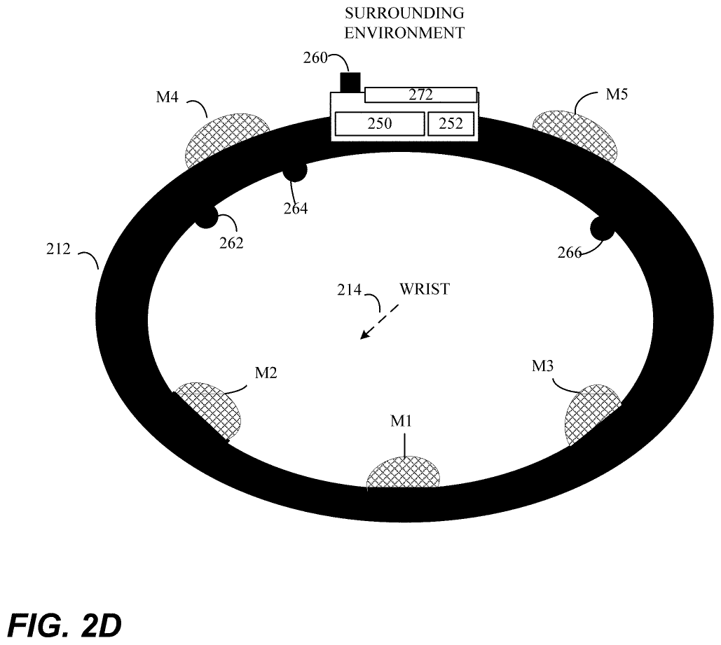

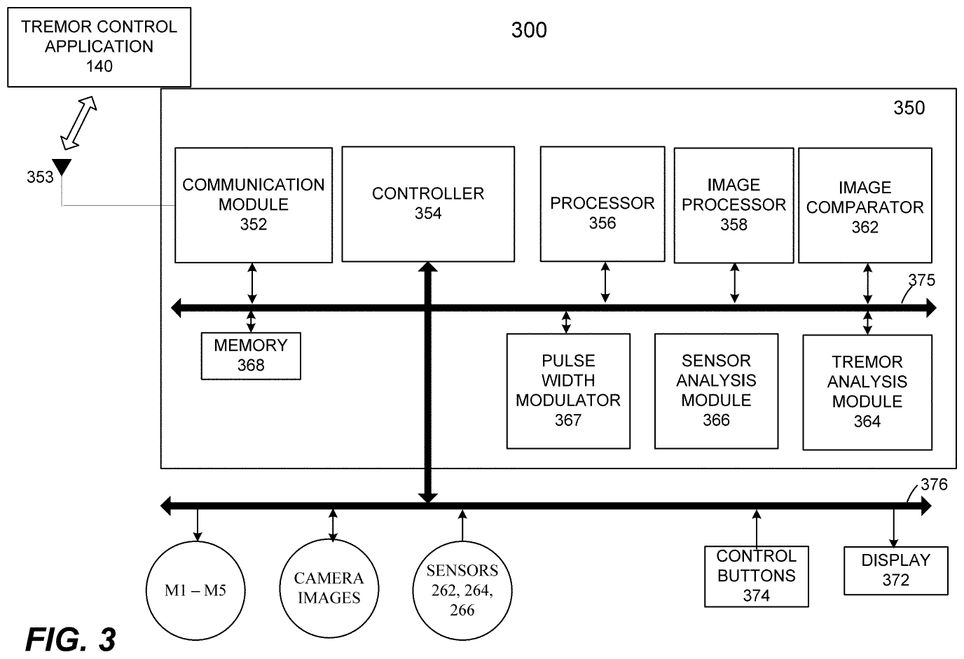

The tremor control application 140 stores a tremor profile of the patient. The tremor occurrences, magnitude and frequencies are added to the tremor profile. The tremor control application processes the tremor occurrences, magnitude and frequencies to determine the tuning parameters of the stimulation needed to offset the tremors. The tremor control application transmits (see arrow 115B) the tuning parameters to the onboard processor 150 of the smart watch, which actuates motors (FIG. 2A, 2B) to generate tremor mitigating mechanical stimulation and noise.

The tuning parameters may include any of motor amplitudes, frequency range, duration of stimulation, patterns of motor actuation, periods of operation, the time of day for monitoring tremors, a schedule for monitoring tremors, or the like.

The tremor mitigating mechanical stimulation is random within the frequency range. Randomness is generated to trick the brain by creating a “small chaos” stimulation in the location of the watch. The random stimulation distracts the brain which stops commanding the hand to move (tremor).

Tremor monitoring may be conducted during daylight hours and/or nighttime hours. The onboard camera captures images (local controller), in a period of 10 images per second. The tremors are detected based on the differences in the images (due to vibration). If tremors are detected, an onboard communication system retrieves the “best known tuning” from the tremor profile in the cloud and transmits the “best known tuning” to the onboard controller to instruct the smart watch to turn on the motors. The tremor profile includes the tuning that was learned from previous tremor events. The tremor profile includes instructions for operating each motor.

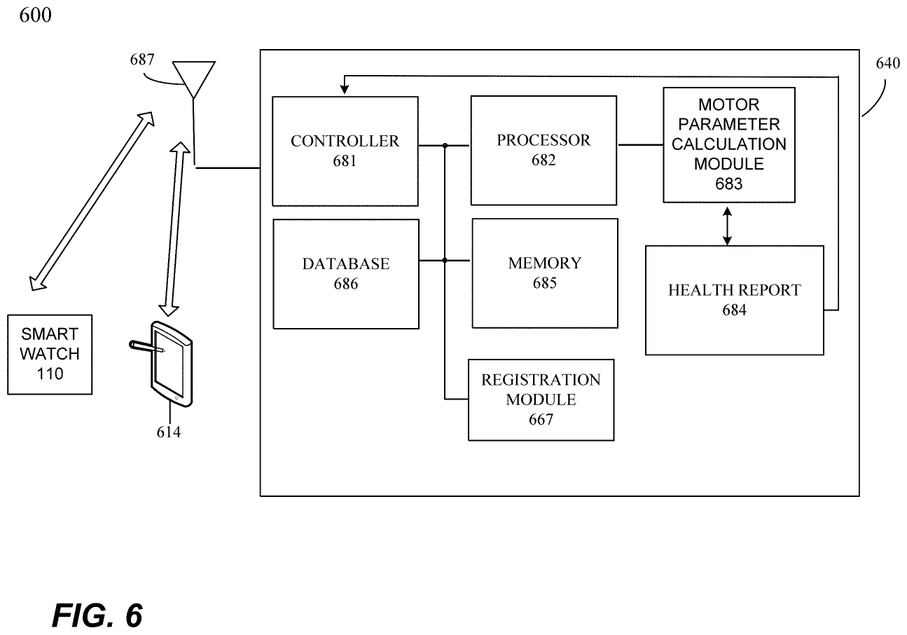

In a non-limiting example of tremor mitigation for patient A, 60% tremor mitigation has been previously achieved using motors M1 and M2, with motors M3-M5 off. This profile is stored in the tremor profile in the tremor application in the cloud. Upon detection of a tremor, the tremor profile is downloaded from the tremor application by the smart watch. The tremor profile may include frequencies for motors M1 and M2 which may be randomly generated by a random number generator in the smart watch controller 354. Each motor may vibrate in the range of 0-12000 rpm. If a reduction of tremor is achieved which is close to 60% (close is defined as at most 10% below the historical best reduction which is saved in the tremor profile in database 686 of the tremor control application 640, FIG. 6), the smart watch motors stay on for 5 minutes. If the reduction of tremor does not approach the historical best tremor reduction of 60%, the tremor analysis module in the smart watch with utilize the other motors. These other motors generate chaotic vibrations intended to distract the brain so that it stops moving the patient's hand.

The smart watch 110 may be configured with an onboard processor and communications module. Alternatively, a first smart watch band may include cameras and sensors and a Bluetooth communications module which transmits the measurements to a second smart watch band for tremor analysis and transmission to the tremor control application. In another alternative, a first smart watch or second smart watch may be designated as a cluster head which transmits the measurements to the tremor control application. The cluster head may be chosen to be the smart watch which has the highest remaining battery power. In a further alternative, the smart watch 110 may transmit the measurements by Bluetooth to a smart device, such as a tablet, smartphone or personal computer of the patient for processing and transmission to the tremor control application 140 in the cloud computing platform environment 130 as shown in FIG. 1.

The cloud computing platform 130 may be connected to the onboard communication module 352 through a network. The network can be a public network, such as the Internet, or a private network such as a local area network (LAN) or a wide area network (WAN) network, or any combination thereof and can also include a public switched telephone network (PSTN) or integrated services for digital network (ISDN) sub-networks. The network may be wireless such as a cellular network including EDGE, 3G 4G, and LTE/LTE-A wireless cellular systems. The wireless network can also be Wi-Fi, Bluetooth, or any other wireless form of communication that as is conventionally known.

Cloud computing is network-based computing in which typically large collections of servers housed in data centers or “server farms” provide computational resources and data storage as needed to remote end users. Some cloud computing services provide access to software applications such as word processors and other commonly used applications to end users who interface with the applications through web browsers or other client-side software.

An application execution system that executes online web applications can implement a platform for distributing web applications. These web applications can be developed on the application execution system and distributed through an online store. Distributed web applications can be installed on accounts so that user data of users that access the installed web application can be segregated from access by developers of the web application, and source code of the web application can be segregated from access by users or purchasers of the web application.

The tremor control software application 140 is deployed on the cloud computing platform 130. The cloud computing platform includes access to storage systems, databases, analytics programs, as needed, that can provide functionality that is required by the tremor control application.

FIG. 2A, 2B illustrate the placement of the tremor modifying motors on a smart watch band 112. The smart watch band hold motors M1, M2 and M3 against the inner wrist of the patient and motors M4 and M5 against the outer wrist of the patient.

The motors M1-M3 are vibration motors. In a non-limiting example, the motors may be Hxchen DC 3V Mini vibration motors 12000 rpm 1030 flat coin button type vibrating motors, 10 mm×3 mm, such as are available from Amazon, https://www.amazon.com

Motors M1 and M2 may have different operating frequency ranges. These are activated based on feedback from the camera. If tremor is not reduced by 50%, the third motor, M3, is activated with a different frequency than motor M1 and M2. Motors M4 and M5 are configured to emit audible noise in frequencies which are effective in reducing tremors.

In a non-limiting example, the motors M4 and M5 may be of the type Part Number 28831, Vibration Motor, which vibrates but also produces noise up to 50 dB, which is the level of noise heard in normal conversation. This type of motor is available from MicroController Pros LLC, 3662 Cindy's Trail, Carson City, Nev., U.S.A. FIG. 2C shows motors of the type which may be used as the motors of the present disclosure.

The number of motors is not limited to five. A greater number of motors can be included on the band, constrained only by the maximum number of motors that are deemed tolerable by the patient and the sizes of the motors.

The onboard processor 250 is included on a circuit board between motors M4 and M5. A camera 260 is also located between motors M4 and M5 with its lens pointed in a direction outwardly from the axis of the band and wrist.

FIG. 2D shows a cross-sectional view of the smart watch showing the placement of the onboard processor 250, camera 260, display 272 and motors M1-M5. The display may show messages such as “tremor onset”, “image acquisition”, “time”, “motors on”, etc. as needed to notify the patient of the operation of the smart watch. The display may also be configured to show the temperature, O₂ level and heart rate of the patient. The wearable band 212 surrounds an extremity, such as the wrist of the patient. The center axis of the wrist is illustrated by dotted line 214. The band may also hold additional sensors, such as body temperature sensor 262 and heart rate sensor 266 and a jack 264 for an O₂ sensor. All wiring for the sensors and motors is embedded in the band 212 and connected to the onboard processor 250.

The wearable band may be constructed of silicone or nylon. The motors are embedded in the band so as to contact the extremity (such as a wrist or an ankle). Motors M1, M2 and M3 project towards the inner wrist or extremity by a height in a range of one to three millimeters, and are preferably located as to touch the wrist at the front of the hand. Motors M4 and M5 project outward from the band towards the back of the wrist or extremity by a height in a range of one to three millimeters and are preferably located to contact the back of the wrist and project outward from the band by three to five millimeters, so as to project vibrations towards the wrist and the ears of the patient. Motors M1, M2 and M3 are spaced equally apart on the inner wrist on one third of the circumference of the band in an arc of from 120-90°, preferably 110-100° measured from a center point of the band when position on a user or when disposed in a circle. Motor M4 and M5 are spaced equally from either side of the circuit board, which is located on one third of the circumference of the band which covers the outer wrist outside the 120-90° arc defining the disposition of motors M1, M2 and M3. Motors M4 and M2 are spaced apart by one sixth of the circumference of the band. Motors M5 and M3 are spaced apart by one sixth of the circumference of the band. The actual circumference of the band depends on whether it is worn on the wrist or the ankle and the size of the patient. In a non-limiting example, for an adult size wrist band 18 cm. in circumference, motors M1, M2 and M3 are on a 6 cm. section of the band and center to center spaced apart about by 2 cm, motors M4 and M5 are spaced apart by 2 cm. from the circuit board and motors M2 and M4 are spaced apart by three cm. The sensors are embedded in the band flush with the wrist and next to or between the motors. A circuit board embedded in the band includes the computing system and the onboard communications module. A display 272 is placed above the circuit board facing outward from the extremity, so that it is easily readable by the tremor patient. The camera 260 is connected to the circuit board with its lens facing outward from the extremity so as to take images of the surrounding environment.

A pulse oximeter records oxygen saturation (O₂) and is a separate device which is clamped over a fingertip. A cord may include a probe which connects into the jack 264 for connection to the smart watch 110. In a non-limiting example, the pulse oximeter may be the 8000AA Reusable Pulse Oximeter Probe available from Concord Health Supply, 9052 Terminal Av, Skokie, Ill., U.S.A.

In a non-limiting example, the body temperature sensor 262 may be an Amphenol Advanced Sensors RL0503-5820-97-MS thermistor, manufactured by Allied Electronics, Inc., 7151 Jack Newell Blvd. S., Fort Worth, Tex. 76118 U.S.A.

In a non-limiting example, the heart rate sensor 266 may be a ROHM Semiconductor BH1790GLC Optical Heart Rate Sensor, available from Mouser Electronics, 1000 North Main Street, Mansfield, Tex., U.S.A.

The camera 260 captures images of the surrounding environment. Combined with techniques such as image subtraction, tremors can be quantified by the onboard processor 250.

In a non-limiting example, the camera 260 may be of the type NanEye miniature CMOS image sensor, manufactured by AMS AG, Tobelbader Strasse 30, 8141 Premstaetten, Austria.

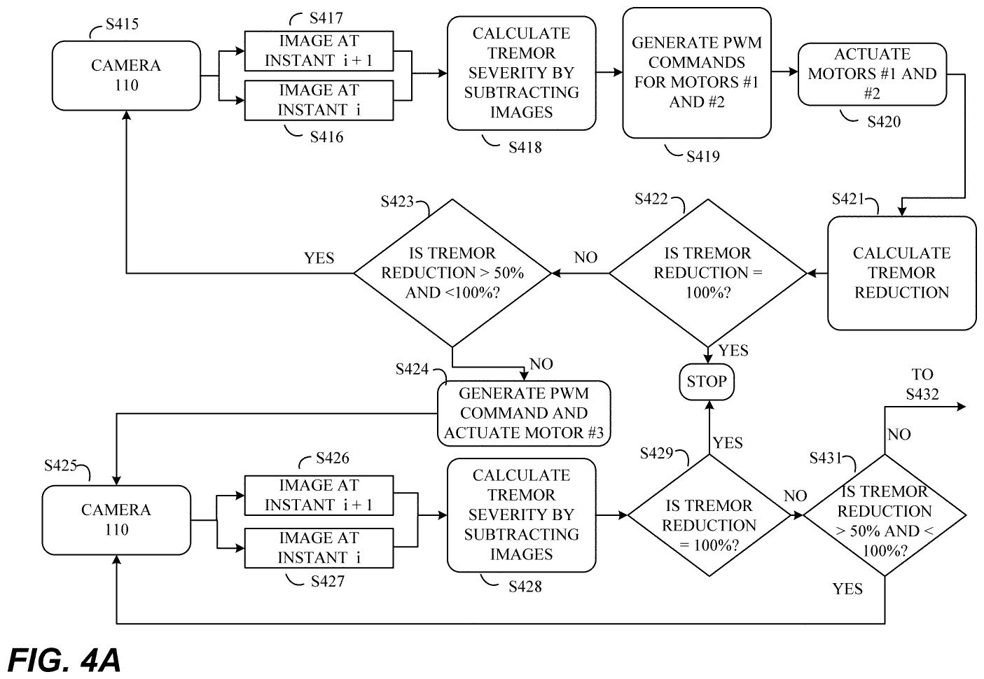

FIG. 3 illustrates the computing system 350 on the circuit board of the smart watch. Onboard communications module 352 is configured for transmitting the tremor occurrences, magnitude and frequencies of the tremors and sensor measurements to the tremor control application.

These measurements allow the detection of the magnitude and phase of tremor, as well as the classification of the type of activity the user is performing (running, walking, sleeping etc.) by the tremor control application 140. The signals from sensors 262, 264 and 266 are processed in sensor analysis module 366 to determine the body temperature, O2 level and heart rate of the patient. These measurements are then compared to profile data stored in a database of the tremor control application and are used in the determination of the motor tuning parameters. Additionally, the body temperature, O₂ level and heart rate may be displayed on display 372 on the smart watch.

The camera images are received at input/output bus 376. The image processor 358 receives the camera images and timestamps the images. The image comparator 362 subtracts each successive image from the one immediately previous to it. If there is a difference between the images, a tremor may be indicated. The tremor analysis module 364 receives the comparisons and determines the magnitude and frequencies of the differences, compares the magnitude and frequencies to thresholds, and determines whether a tremor has occurred.

The magnitude and frequencies that indicate tremors and the sensor analyses of temperature, O₂ and heart rate are transmitted to the controller over bus line 375. The controller concatenates the measurements with an identity of the smart watch, stored in memory 368, to form a communications packet which is transmitted by the onboard communications module 352 to the tremor control application 140.

The tremor control software application 140 receives the data packet, updates a body profile database with the information and uses the measurements to determine the magnitudes and frequency ranges for each motor M1-M5. These magnitudes and frequency ranges are transmitted to the onboard communication system 310 and are used by controller 354 to actuate the motors M1-M5 by pulse width modulator 367. Control buttons 374 may be included on the band 212 or on the display 372 to turn the monitoring ON and OFF, to set time, date and time zone for use by the image processor 358 in generating timestamps for the images.

The controller 354 is communicatively coupled to the motors, processor 356, image processor 358, image comparator 362, sensors 262, 264, 266, display 372, tremor analysis module 364, sensor analysis module 366, memory 368, and camera 260 to receive/send data therefrom, for example, via bus lines 375, 376. Further, the controller 354 is communicatively coupled to the onboard communication module 352 to transmit and receive communications to or from tremor control application in the cloud 130.

The control system 350 may comprise a single Central Processing Unit (CPU), or could comprise two or more processing units. For example, the processing unit 356 may include general purpose microprocessors, instruction set processors and/or related chips sets and/or special purpose microprocessors such as Application Specific Integrated Circuits (ASICs). The processing unit 356 may also comprise a memory or storage for caching and other purposes.

Principal components of a processor include an arithmetic logic unit (ALU) that performs arithmetic and logic operations, processor registers that supply operands to the ALU and store the results of ALU operations, and a control unit that orchestrates the fetching (from memory) and execution of instructions by directing the coordinated operations of the ALU, registers and other components.

The memory 368 is a computer readable medium and is connected to the controller 354 and processor 356. The controller 354 and processor 356 may be embodied in a single unit, as a CPU. The memory stores computer readable instructions e.g. in the form of computer program modules. For example, the memory 368 may be a flash memory, a Random-Access Memory (RAM), a Read-Only Memory (ROM) or an Electrically Erasable Programmable ROM (EEPROM).

Referring to FIG. 6, the tremor control application includes a registration module 667 for registering a user with the tremor control application. Registration may entail downloading the tremor control application to a smart computing device of a user. Registration comprises the creation of public and private pair keys by the user, and transmitting the public key (included in the log in credentials) to the tremor control application. Registration with the tremor control application may be free or may require a subscription fee. Access to the tremor control application may be provided with the purchase of a smart watch or may require a separate subscription fee.

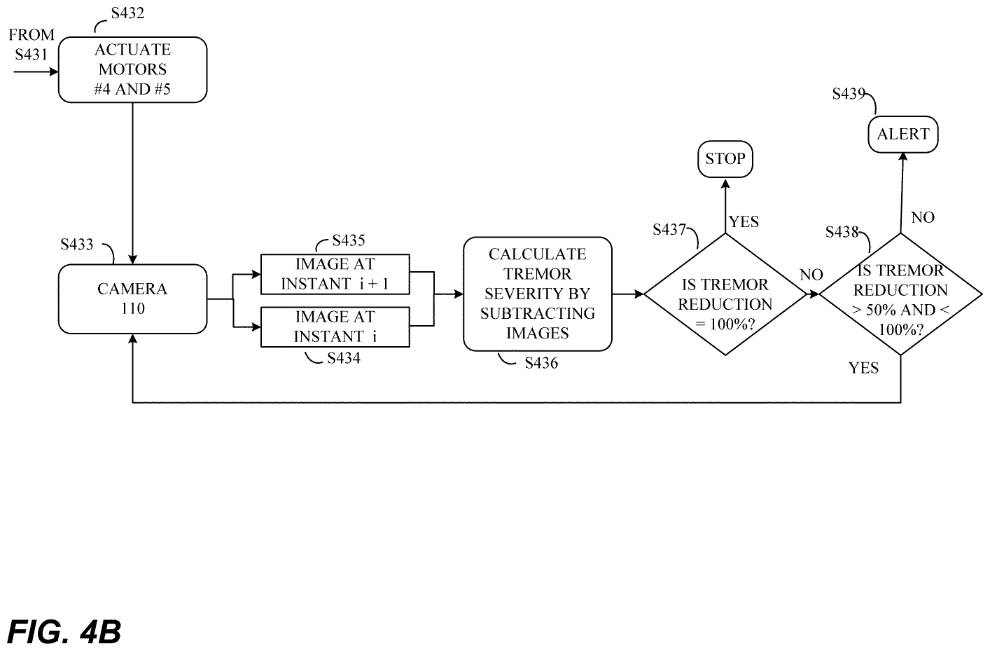

FIG. 4A and FIG. 4B are a flowchart illustrating processing of the images by the tremor analysis module 364 once the motor frequencies and amplitudes have been received from the tremor control application 140. The goal of the process is to reduce the tremor severity by more than 50%. If the tremors cannot be reduced, an alert is generated. The alert may be displayed on the smart watch display 372 or may be sent to the tremor control application, where a physician or designated person or authority is notified. At S415, the camera begins taking images of the surrounding environment every “i” seconds. The initial value of “i” may be a fraction of a second. “i” represents a time increment which is determined by the type of tremor monitored. For example, for a tremor frequency of 5 Hz, a time of one second represents 5 cycles of the tremor. In this situation, “i” may be 0.02 seconds, which monitors each tremor five times per cycle. At step 416, the image is taken at “i” seconds and at S417, an image is taken at “I+1” seconds. These two images are subtracted at S418 and the tremor severity is determined by the magnitude of the difference. A succession of differences is used to calculate the frequency of the tremor. At S419, the frequency of the tremor and the magnitudes are stored and PWM commands are generated. At S420, the PWM command is used to actuate the motors M1 and M2. At S421, several cycles are compared and the tremor reduction is calculated. At S422, if the tremor reduction is 100%, i.e., if the tremors have stopped, then the process stops. If the tremor reduction is greater than 50% but less than 100%, the images are again monitored at S415. If the tremor reduction is below 50%, then motor M3 is actuated at S424, the camera takes images at S425, 426, 427 and the tremor severity is recalculated at S428. At S429, if the tremor reduction is 100%, the process stops. If NO, if the tremor reduction is greater than 50% but less than 100%, the images are again monitored at S425. If the tremor reduction is below 50%, the process proceeds to S432 of FIG. 4B. At S432, motors M4 and M5 are actuated to generate both vibrations and audible noise. The camera 110 again begins to take images at S433. The image at instant i and the image at instant i+1 are acquired for a succession of images in steps S434 and S435. At S436, the tremor severity is then calculated by subtracting images, determining the magnitudes of the differences and determining the frequency of the tremor. At S437, if the tremor reduction equals 100%, the process stops. If the tremor reduction is greater than 50% and less than 100%, the process returns to S433 and continues to analyze the tremors. If there is no improvement in the tremors, an alert is sent at S439 and displayed on the display 372.

The images taken by the camera of the surrounding environment are used to identify a tremor. A tremor causes a hand to shake, thus the images taken by the camera of the surrounding environment will differ. A series of timestamped images will show the frequency and magnitude of the tremors when the images are compared. Subtraction of each successive image from a previous image gives the magnitude of the tremor. Analysis of a series of successive images or of the successive differences yields the frequency of the tremors. The comparison of the images is performed by image comparator 362. In a non-limiting example, the image comparator may include software to determine the differences between images, such as ImageDiff, which is a pixel by pixel image comparison tool, which compares two images side by side, and generates a difference mask showing the exact pixels that have changed. (See: “Image Diff”, Free Download, ionForge, published online, Jun. 28, 2007, http://www.ionforge.com.)

The image frames are read every 0.05 seconds. Each image is turned into a grayscale image. Pixels of the grayscale image at time frame (i) are subtracted from corresponding pixels of the gray image at time frame (i−1). The image subtraction may focus on a reference feature in the image and subtract only the pixels around the reference feature. A reference feature may be detected by a difference in pixel intensity or density of the reference feature as compared to the background.

The differences between the images are summed and divided by the image size (dimensions of the image or the image quality=width×height). In a non-limiting example, the patient may be sitting in a room and the image may be a view of the room as seen from the wrist. Features, such as doors, windows or artwork in the image may provide reference points for the image subtraction. Images can be recorded in any of the following three sizes: large (L), medium (M), and small (S). In a non-limiting example, a large size image may be equivalent to about 16 megapixels, a medium size image to about 9 megapixels, and a small size image to about 4 megapixels. Since images are taken every 0.05 seconds, the patient may move, walk, run or sit without affecting the view of the reference point, as tremors of frequency 5-8 Hz repeat every 5-8 seconds and the image acquisition is one twentieth of a second.

The threshold for the difference in images is [−5, 5]. If the maximum amplitude of difference in the images falls outside these ranges, then tremor is detected.

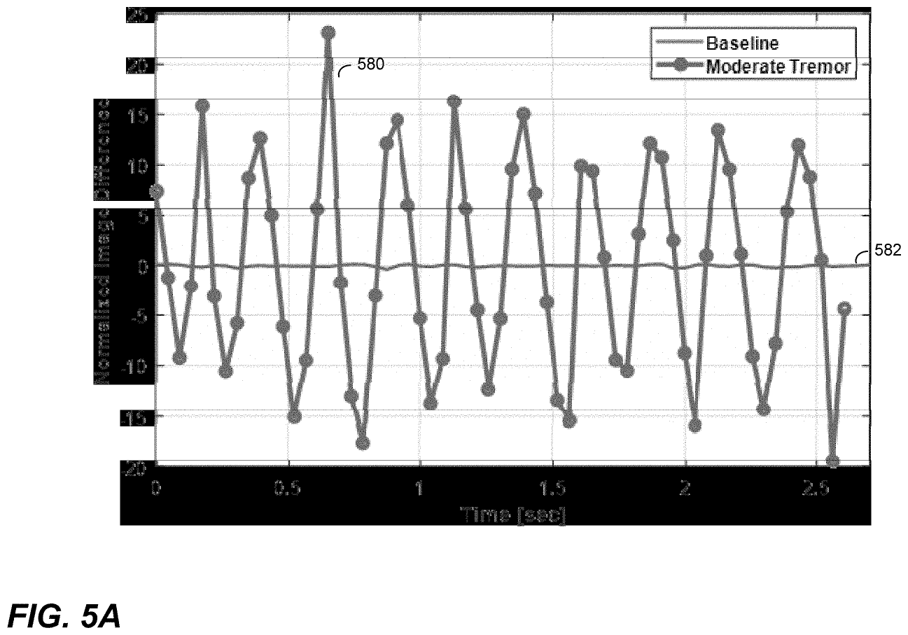

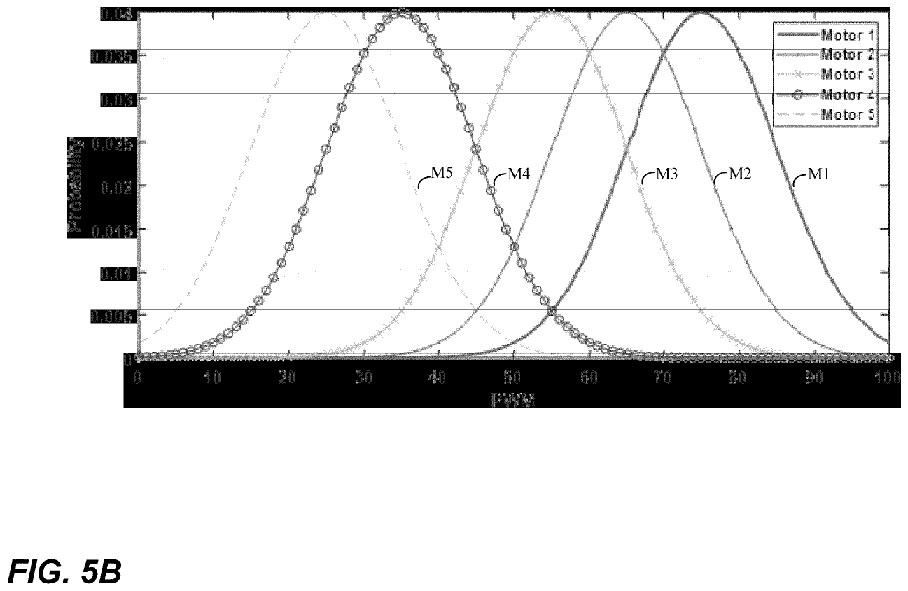

FIG. 5A depicts the normalized difference in the images plotted as function of time for the case of no tremor (baseline 582) and moderate tremor (curve 580).

The motors are driven via pulse width modulation by pulse width modulator 367. At a command for 100% frequency, the motor will vibrate at 12000 rpm. A command of 0% turns the motor off. A command of 0-100% power is sent to each motor every 0.1 seconds. The following is a list of the commands to each motor:

Motor 1: the command is a randomly generated number (normal distribution) of mean of 75%, and standard deviation of 10%

Motor 2: the command is a randomly generated number (normal distribution) of mean of 65%, and standard deviation of 10%

Motor 3: the command is a randomly generated number (normal distribution) of mean of 55%, and standard deviation of 10% Motor 4: the command is a randomly generated number (normal distribution) of mean of 45%, and standard deviation of 10%

Motor 5: the command is a randomly generated number (normal distribution) of mean of 35%, and standard deviation of 10%.

FIG. 5B shows the normal distribution for the PWM signal for each motor. These distributions are saved in a form of table data, where the PWM of each motor is generated randomly.

Motor 1 is located between M2 and M3 and as seen from the distribution, is the motor that has the maximum mean (it is the most likely to vibrate at the highest PWM frequency).

When a random number is generated in the controller 354, a value is randomly generated for pulse width modulation of M1. For example, M1 may receive a value of 50%, which means its commanded pulse width modulation will be 50% for an execution time of 0.1 second.

In the next execution, the random number that is generated in the controller may be 70% for M1, which represents a command of 70% for the PWM.

FIG. 6 illustrates the computing environment of the tremor control application (140, FIG. 1, 640, FIG. 6. The tremor analysis results (computations of reduction in severity at S422, S423, S429, S431, S437 and S438), as well as login credentials, magnitude and frequencies from the camera image analysis that indicate tremors and the sensor analyses of temperature, O2 and heart rate are transmitted as a data packet from the smart watch onboard communications module 352 to the tremor control application 640. The data packet sent may contain log in credentials that can be used to determine permissions within the database 686 and to determine where in the database the information should be saved.

Program code in the tremor control application may be implemented to track changes in the images. The program code may be developed, instrumented or embedded for execution on a general or specialized computing machine. The program code is designed as software (hereafter referred to as a computer application or “app” by way of example) which “app” also may be installed on the onboard processor of the smart watch or on handheld user device, such as a tablet or a smart phone.

The cloud server 130 can represent one or more servers communicably coupled to the on-board communication module 352. For example, the server can include processing circuitry configured to operate the system 100, receive data from the onboard communication module 352, receive statistical information from the database 686 and the like. The server may include an application server which hosts a web application which performs some or all of the processes of the tremor control application. The server may include a communication endpoint or find other endpoints and communicate with those endpoints. The server may share computing resources, such as CPU and random-access memory over a network. The server may be a virtual server or a web server. The cloud network enables the communication between the on-board communication module, satellites or base stations and tremor control application.

The processing circuitry of the tremor control application 640 residing on the server can carry out instructions to perform or cause performance of various functions, operations, steps or processes of the system 100. The controller 681 and processing circuitry 682 can be configured to store information in memory, operate the system 100, and receive and send information in the form of signals between the components of the tremor control application.

The tremor control application 640 receives the data packet from the onboard communications module 352 of the smart watch 110. The log in credentials are matched to log in credentials stored in the database 686. The patient's profile is retrieved and the database is updated with the new measurements.

The tremor analysis results (computations of reduction in severity at S422, S423, S429, S431, S437 and S438), as well as magnitudes and frequencies that indicate tremors are input to the motor parameter calculation module 683, which calculates amplitudes and frequency ranges for the motors M1-M5. The motor parameter calculation module may also tailor patterns of motor operation, amplitudes and frequencies to reduce the tremors based on the previous results.

A health report module 684 receives the tremor analysis results, magnitudes and frequencies that indicate tremors, the sensor analyses of temperature, O2 and heart rate and generates a health report. The health report may be transmitted to a user device 614, such as a tablet, computer or smart phone of the patient. The health report may include graphs or statistics showing the heart rates, O2 levels and temperatures of the patient over time. The health report may further show graphs or statistics regarding tremor severity and the effectiveness of the smart watch vibratory treatment.

The first embodiment is illustrated with respect to FIGS. 1, 2D, 3, 4A, 4B and 6. The first embodiment describes a smart watch 110 for tremor monitoring and control of an extremity of a user, comprising a wearable band 212 including a camera 260, a plurality of motors (M1-M5), an onboard communications module 252 configured to communicate with a cloud server 130 storing a tremor control application 140, and a computing system (250, 350) including circuitry and a processor 356 having program instructions configured to actuate the camera to take a series of images, generate an analysis of the series of images and actuate the motors to generate tremor control vibrations based on the analysis of the series of images.

The computing system is operatively connected to the camera, the plurality of motors, and the onboard communications module. The computing system is further configured to monitor a tremor reduction effect of the vibrations, generate data packets including the analysis of the series of images and the tremor reduction effect, and wherein the onboard communications module is configured to transmit the data packets (along communications path 115A) to the tremor control application.

The wearable band further comprises at least one of a plurality of sensors operatively connected to the computing system, the plurality of sensors including at least one of a body temperature sensor 262, an O₂ pulse oximeter 264, a heart rate monitor 266, wherein the computing system is configured to receive at least one of a temperature, O₂ and heart rate measurement from the plurality of sensors and include the measurement in the data packets sent to the tremor control application.

The plurality of motors includes a set of motors which vibrate without emitting audible sound (M1, M2, M3) and a set of motors which vibrate and emit audible frequencies (M4, M5).

The computing system further includes a memory 368 storing log in credentials, wherein the computing system is further configured to include the log in credentials in the data packet sent to the tremor control application.

The tremor control application 600 includes a transceiver 687 configured to receive the data packets, a registration module 667 configured to register a computing device 614 of the smart watch user with the tremor control application and generate a unique access code, an application memory 685 configured to store unique access codes of a plurality of smart watch users registered with the tremor control application, a database 686 configured to store a body profile including a tremor profile of each smart watch user, application circuitry 681 and an application processor 682 having program instructions configured to match the log in credentials to one of the unique access codes, retrieve the body profile of the user associated with the unique access code, update the database with the at least one sensor measurement, the analysis of the series of images and the tremor reduction effect, compare the at least one sensor measurement, the analysis of the series of images and the tremor reduction effect to the body profile, identify an onset of tremors, detect magnitudes and frequencies of the tremors, generate motor tuning parameters (motor parameter calculation module 683) to reduce the tremors, send the motor tuning parameters to the transceiver, wherein the transceiver is further configured to transmit the motor tuning parameters (along communication pathway 115B) to the onboard communications module of the smart watch.

The tremor control application further comprises a health report module 684 configured to receive the at least one sensor measurement, the body profile and the tremor reduction effect and generate a health report, wherein the transceiver 686 is further configured to transmit the health report to at least one of the onboard communications module 352 and the computing device 614 of the smart watch user.

The motor tuning parameters include an amplitude and frequency ranges for each motor of the smart watch. The motor tuning parameters may optionally include one or more of a pattern of motor actuation, a period of operation, time of day for monitoring tremors, a schedule for monitoring tremors.

The onboard communications module is further configured to receive the motor tuning parameters, wherein the computing system further includes a pulse width modulator 367 configured to generate commands for each motor of the plurality of motors, wherein the circuitry is configured to use the commands to actuate each motor according to the motor tuning parameters.

The second embodiment is illustrated with respect to FIGS. 1, 2D, 3, 4A, 4B and 6. The second embodiment describes method for monitoring and controlling tremors in an extremity of a patient by a smart watch 110, comprising actuating a camera 260 located on the extremity to acquire a first series of images of an environment surrounding the extremity, analyzing the first series of images to determine a magnitude and frequency of a tremor, actuating at least one of a plurality of motors (M1-M5) located on the extremity to generate tremor control vibrations based on the analysis of the first series of images.

Analyzing the first series of images comprises timestamping the images, subtracting each image from an image having an immediately previous timestamp to generate a time series of differences, normalizing the differences, comparing a magnitude of each difference to a threshold, identifying a tremor occurrence if the magnitude is greater than the threshold, and determining a frequency of the tremors by mapping the tremor occurrences with respect to the timestamps.

The method further comprises retrieving log in credentials from a memory 368 of the smart watch, transmitting the log in credentials, magnitudes and frequencies in a data packet to a tremor control application 140, receiving the data packet by the tremor control application (by transceiver 687), matching the log in credentials to a database 686 of access codes of the tremor control application, retrieving a body profile of the patient from the database, generating, by the tremor control application (in motor parameter calculation module 683), motor tuning parameters based on the magnitudes, frequencies and the body profile, transmitting the motor tuning parameters to the smart watch 110, receiving, by the smart watch, the motor tuning parameters, and generating commands by pulse width modulation (see pulse width modulator 367) to actuate each motor according to the motor tuning parameters.

The method includes registering the smart watch with the tremor control application by creating public and private pair keys by the smart watch, including the public key in the data packet with the log in credentials, transmitting the data packet to the tremor control application and storing the public key as an access code in the database.

The method includes registering a computing device 614 of the patient with the tremor control application by transmitting the public key to the tremor control application by the computing device, matching the public key to the access code, creating a body profile including at least one of age, height, previous tremor occurrence magnitudes and frequencies, previous tremor mitigation strategies, gender, ethnic group, address, credit card information and medical diseases of the patient, and generating a contact list including at least one of a preferred medical practitioner, a next of kin, a hospital, a medical alert center and a contact person.

As shown in FIGS. 4A and 4B, the method includes monitoring a first tremor reduction effect due to the tremor control vibrations generated by a first motor and a second motor, comparing the first tremor reduction effect to a threshold, stopping the first and second motors if the first tremor reduction effect is 100%, continuing to actuate the first and second motors if the first tremor reduction effect is greater than 50% and less than 100%, actuating a third motor located between the first and second motor if the first tremor reduction effect is less than or equal to 50%, monitoring a second tremor reduction effect due to the tremor control vibrations generated by the first, second and third motors, comparing the second tremor reduction effect to the threshold, stopping the first, second and third motors if the second tremor reduction effect is 100%, continuing to actuate the first, second and third motors if the second tremor reduction effect is greater than 50% and less than 100%, actuating fourth and fifth motors located opposite the first and second motors respectively to generate vibrations and audible frequencies if the second tremor reduction effect is less than or equal to 50%, monitoring a third tremor reduction effect due to the tremor control vibrations generated by the first, second, third, fourth and fifth motors and the audible frequencies generated by the fourth and fifth motors, comparing the third tremor reduction effect to the threshold, stopping the first, second, third, fourth and fifth motors if the tremor reduction effect is 100%, continuing to actuate the first, second, third, fourth and fifth motors if the tremor reduction effect is greater than 50% and less than 100%, sending an alert to a contact person on the contact list if the third tremor reduction effect is less than or equal to 50%, transmitting the first, second and third tremor reduction effects to the tremor control application and storing the first, second and third tremor reduction effects in the database with the body profile.

The method further includes receiving, by a health report module 684 of the tremor control application, a sensor measurement from at least one of a plurality of sensors located on the smart watch, wherein the plurality of sensors includes a body temperature sensor 262, an O₂ pulse oximeter 264 and a heart rate monitor 266, receiving, by the health report module, the body profile and first, second and third tremor reduction effects, correlating the at least one sensor measurement, the body profile and first, second and third tremor reduction effects, generating a health report, and transmitting the health report to at least one of the onboard communications module 352 and the computing device 614 of the patient.

The third embodiment is illustrated with respect to FIGS. 1, 2D, 3, 4A, 4B and 6. The third embodiment describes a smart watch system for monitoring and controlling tremors in an extremity of a patient, comprising a wearable band 212 surrounding the extremity, the wearable band including a camera 260 configured to acquire a first series of images of an environment surrounding the extremity, an image processor 358 configured to receive the first set of images and timestamp each image, an image comparator 362 configured to subtract each image from an image having an immediately previous timestamp to generate a time series of differences, a tremor analysis module 364 configured normalize the differences, compare the magnitude of each difference to a threshold, identify a tremor occurrence if the magnitude is greater than the threshold and determine a frequency of the tremors by mapping the tremor occurrences with respect to the timestamps, a plurality of sensors including at least one of a body temperature sensor 262, an O2 pulse oximeter 264 and a heart rate monitor 266, the plurality of sensors configured to take measurements of the extremity, a plurality of motors (m1-M5) configured to generate at least one of vibrations in the extremity and vibrations in the extremity and audible frequencies, a pulse width modulator 367 configured to generate commands to actuate the motors to generate the vibrations, a memory 368 storing log in credentials, a computing system 350 including circuitry (controller 354) and a processor 356 having program instructions configured to actuate the camera to acquire the first series of images, instruct the tremor analysis module to determine the frequency and magnitude of the tremors, actuate at least one of the plurality motors to generate tremor control vibrations based on the analysis of the series of images, monitor a first tremor reduction effect due to the vibration of the at least one motor, generate a data packet including the at least one sensor measurement, the magnitude and frequency of the tremor occurrence, the first tremor reduction effect and the log in credentials, an onboard communications module 352 configured to transmit the data packet to a cloud server 130, a tremor control application 140 stored on the cloud server, the tremor control application including a transceiver 687 configured to receive the data packets, a registration module 667 configured to register a computing device of the patient with the tremor control application and to generate a unique access code, an application memory 685 configured to store unique access codes of a plurality of smart watch patients registered with the tremor control application, a database 686 configured to store a body profile of the patient, application circuitry (controller 681) and an application processor 682 having program instructions configured to match the log in credentials to one of the unique access codes, retrieve the body profile of the user associated with the unique access code, update the database the at least one sensor measurement, the magnitude and frequency of the tremor occurrence and the tremor reduction effects, compare the at least one sensor measurement, the magnitude and frequency of the tremor occurrence, the tremor reduction effects to the body profile, generate motor tuning parameters to reduce the tremors, wherein the motor tuning parameters include at least an amplitude and frequency range for each motor of the smart watch, send the motor tuning parameters to the transceiver, wherein the transceiver is further configured to transmit the motor tuning parameters to the onboard communications module 352 of the smart watch.

The tremor analysis application 600 further comprises a health report module 684 configured to receive the at least one sensor measurement, the body profile and the tremor reduction effect and generate a health report, wherein the transceiver 687 is further configured to transmit the health report to at least one of the onboard communications module 352 and the computing device 614 of the patient.

The computing system is further configured to monitor an effectiveness of the vibrations, by monitoring a first tremor reduction effect due to the tremor control vibrations generated by a first motor and a second motor, comparing the first tremor reduction effect to a threshold, stopping the first and second motors if the first tremor reduction effect is 100%, continuing to actuate the first and second motors if the first tremor reduction effect is greater than 50% and less than 100%, actuating a third motor located between the first and second motor if the first tremor reduction effect is less than or equal to 50%, monitoring a second tremor reduction effect due to the tremor control vibrations generated by the first, second and third motors, comparing the second tremor reduction effect to the threshold, stopping the first, second and third motors if the second tremor reduction effect is 100%, continuing to actuate the first, second and third motors if the second tremor reduction effect is greater than 50% and less than 100%, actuating fourth and fifth motors located opposite the first and second motors respectively to generate vibrations and audible frequencies if the second tremor reduction effect is less than or equal to 50%, monitoring a third tremor reduction effect due to the tremor control vibrations generated by the first, second, third, fourth and fifth motors and the audible frequencies generated by the fourth and fifth motors, comparing the third tremor reduction effect to the threshold, stopping the first, second, third, fourth and fifth motors if the tremor reduction effect is 100%, continuing to actuate the first, second, third, fourth and fifth motors if the tremor reduction effect is greater than 50% and less than 100%, sending an alert to a contact person on the contact list if the third tremor reduction effect is less than or equal to 50%, transmitting the first, second and third tremor reduction effects to the tremor control application and storing the first, second and third tremor reduction effects in the database with the body profile.

The plurality of motors may equal five motors and the pulse width monitor is configured to generate commands to the plurality of motors in the range of 0-100% power with a standard deviation of 10%, wherein the command to a first motor is a randomly generated number of mean of 75%, a second motor is a randomly generated number of mean of 65%, a third motor is a randomly generated number of mean of 55%, a fourth motor is a randomly generated number of mean of 45%, and a fifth motor is a randomly generated number of mean of 35%.

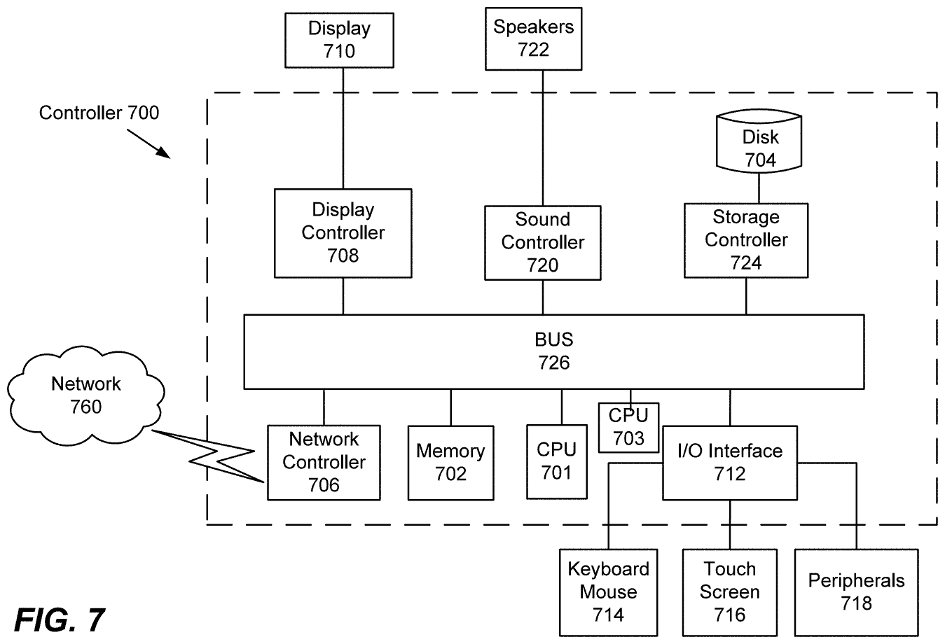

Next, further details of the hardware description of the computing environment of FIG. 6 according to exemplary embodiments are described with reference to FIG. 7. In FIG. 7, a controller 700 is described is representative of the system 600 of FIG. 6 in which the controller is a computing device which includes a CPU 701 which performs the processes described above/below. The process data and instructions may be stored in memory 702. These processes and instructions may also be stored on a storage medium disk 704 such as a hard drive (HDD) or portable storage medium or may be stored remotely.

Further, the claims are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computing device communicates, such as a server or computer.

Further, the claims may be provided as a utility application, background daemon, or component of an operating system, or combination thereof, executing in conjunction with CPU 701, 703 and an operating system such as Microsoft Windows 7, UNIX, Solaris, LINUX, Apple MAC-OS and other systems known to those skilled in the art.

The hardware elements in order to achieve the computing device may be realized by various circuitry elements, known to those skilled in the art. For example, CPU 701 or CPU 703 may be a Xenon or Core processor from Intel of America or an Opteron processor from AMD of America, or may be other processor types that would be recognized by one of ordinary skill in the art. Alternatively, the CPU 701, 703 may be implemented on an FPGA, ASIC, PLD or using discrete logic circuits, as one of ordinary skill in the art would recognize. Further, CPU 701, 703 may be implemented as multiple processors cooperatively working in parallel to perform the instructions of the inventive processes described above.

The computing device in FIG. 7 also includes a network controller 706, such as an Intel Ethernet PRO network interface card from Intel Corporation of America, for interfacing with network 760. As can be appreciated, the network 760 can be a public network, such as the Internet, or a private network such as an LAN or WAN network, or any combination thereof and can also include PSTN or ISDN sub-networks. The network 760 can also be wired, such as an Ethernet network, or can be wireless such as a cellular network including EDGE, 3G and 4G wireless cellular systems. The wireless network can also be WiFi, Bluetooth, or any other wireless form of communication that is known.

The computing device further includes a display controller 708, such as a NVIDIA GeForce GTX or Quadro graphics adaptor from NVIDIA Corporation of America for interfacing with display 710, such as a Hewlett Packard HPL2445w LCD monitor. A general purpose I/O interface 712 interfaces with a keyboard and/or mouse 714 as well as a touch screen panel 716 on or separate from display 710. General purpose I/O interface also connects to a variety of peripherals 718 including printers and scanners, such as an OfficeJet or DeskJet from Hewlett Packard.

A sound controller 720 is also provided in the computing device such as Sound Blaster X-Fi Titanium from Creative, to interface with speakers/microphone 722 thereby providing sounds and/or music.

The general purpose storage controller 724 connects the storage medium disk 704 with communication bus 726, which may be an ISA, EISA, VESA, PCI, or similar, for interconnecting all of the components of the computing device. A description of the general features and functionality of the display 710, keyboard and/or mouse 714, as well as the display controller 708, storage controller 724, network controller 706, sound controller 720, and general purpose I/O interface 712 is omitted herein for brevity as these features are known.

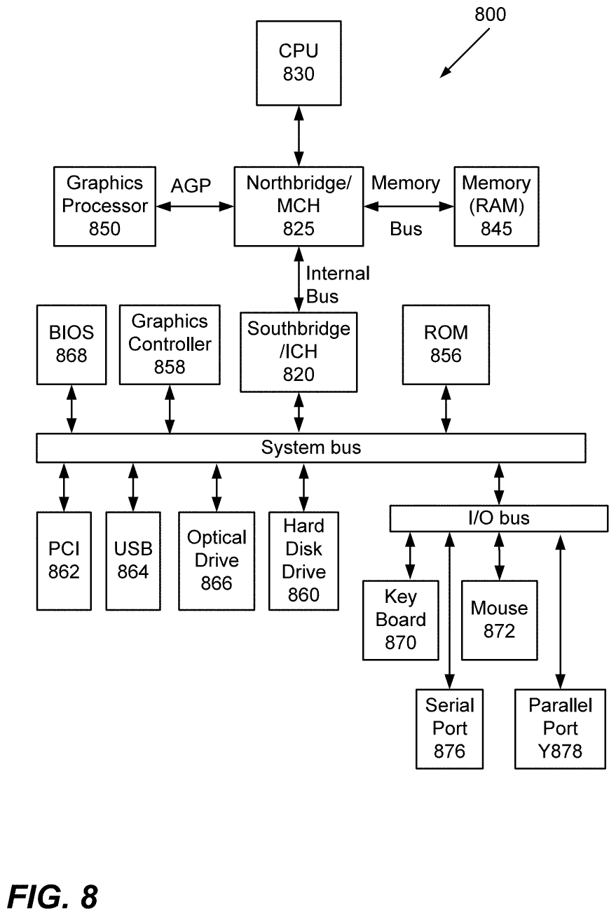

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown on FIG. 8.

FIG. 8 shows a schematic diagram of a data processing system, according to certain embodiments, for performing the functions of the exemplary embodiments. The data processing system is an example of a computer in which code or instructions implementing the processes of the illustrative embodiments may be located.

In FIG. 8, data processing system 800 employs a hub architecture including a north bridge and memory controller hub (NB/MCH) 825 and a south bridge and input/output (I/O) controller hub (SB/ICH) 820. The central processing unit (CPU) 830 is connected to NB/MCH 825. The NB/MCH 825 also connects to the memory 845 via a memory bus, and connects to the graphics processor 850 via an accelerated graphics port (AGP). The NB/MCH 825 also connects to the SB/ICH 820 via an internal bus (e.g., a unified media interface or a direct media interface). The CPU Processing unit 830 may contain one or more processors and even may be implemented using one or more heterogeneous processor systems.

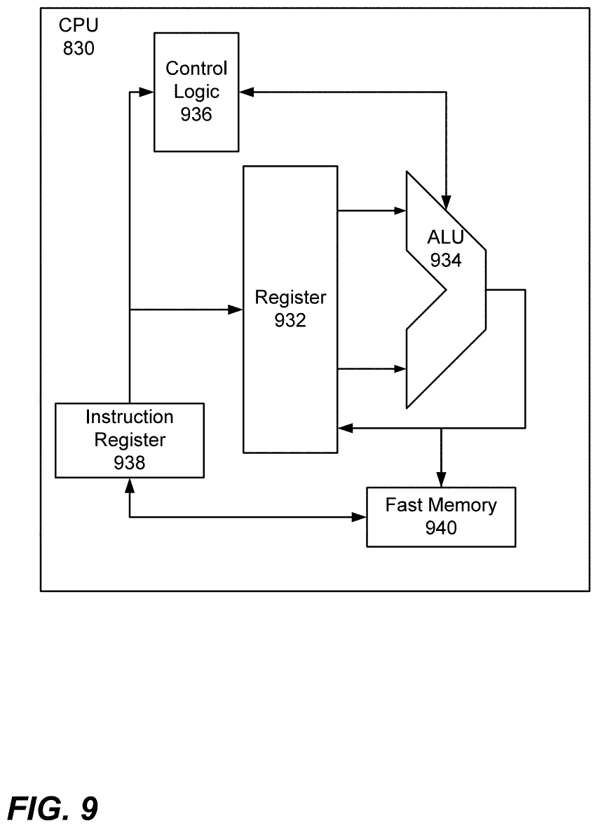

For example, FIG. 9 shows one implementation of CPU 830. In one implementation, the instruction register 938 retrieves instructions from the fast memory 940. At least part of these instructions are fetched from the instruction register 938 by the control logic 936 and interpreted according to the instruction set architecture of the CPU 830. Part of the instructions can also be directed to the register 932. In one implementation the instructions are decoded according to a hardwired method, and in another implementation the instructions are decoded according a microprogram that translates instructions into sets of CPU configuration signals that are applied sequentially over multiple clock pulses. After fetching and decoding the instructions, the instructions are executed using the arithmetic logic unit (ALU) 934 that loads values from the register 932 and performs logical and mathematical operations on the loaded values according to the instructions. The results from these operations can be feedback into the register and/or stored in the fast memory 940. According to certain implementations, the instruction set architecture of the CPU 830 can use a reduced instruction set architecture, a complex instruction set architecture, a vector processor architecture, a very large instruction word architecture. Furthermore, the CPU 830 can be based on the Von Neuman model or the Harvard model. The CPU 830 can be a digital signal processor, an FPGA, an ASIC, a PLA, a PLD, or a CPLD. Further, the CPU 830 can be an x86 processor by Intel or by AMD; an ARM processor, a Power architecture processor by, e.g., IBM; a SPARC architecture processor by Sun Microsystems or by Oracle; or other known CPU architecture.

Referring again to FIG. 8, the data processing system 800 can include that the SB/ICH 820 is coupled through a system bus to an I/O Bus, a read only memory (ROM) 856, universal serial bus (USB) port 864, a flash binary input/output system (BIOS) 868, and a graphics controller 858. PCI/PCIe devices can also be coupled to SB/ICH 888 through a PCI bus 862.

The PCI devices may include, for example, Ethernet adapters, add-in cards, and PC cards for notebook computers. The Hard disk drive 860 and CD-ROM 866 can use, for example, an integrated drive electronics (IDE) or serial advanced technology attachment (SATA) interface. In one implementation the I/O bus can include a super I/O (SIO) device.

Further, the hard disk drive (HDD) 860 and optical drive 866 can also be coupled to the SB/ICH 820 through a system bus. In one implementation, a keyboard 870, a mouse 872, a parallel port 878, and a serial port 876 can be connected to the system bus through the I/O bus. Other peripherals and devices that can be connected to the SB/ICH 820 using a mass storage controller such as SATA or PATA, an Ethernet port, an ISA bus, a LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry, or based on the requirements of the intended back-up load to be powered.

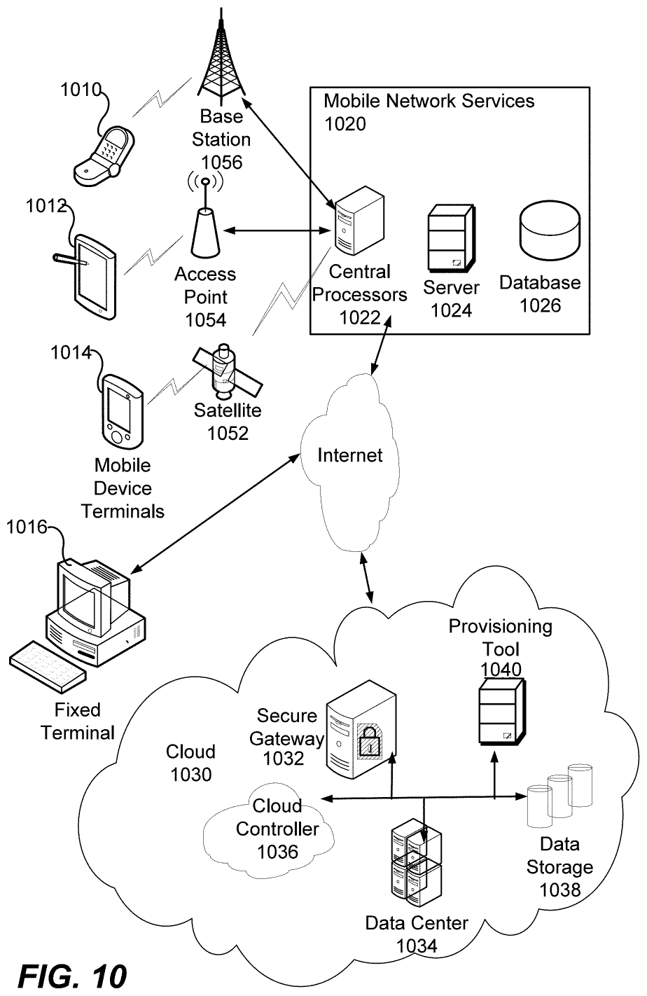

The functions and features described herein may also be executed by various distributed components of a system. For example, one or more processors may execute these system functions, wherein the processors are distributed across multiple components communicating in a network. The distributed components may include one or more client and server machines, which may share processing, as shown by FIG. 10, in addition to various human interface and communication devices (e.g., display monitors, smart phones, tablets, personal digital assistants (PDAs)). The network may be a private network, such as a LAN or WAN, or may be a public network, such as the Internet. Input to the system may be received via direct user input and received remotely either in real-time or as a batch process. Additionally, some implementations may be performed on modules or hardware not identical to those described. Accordingly, other implementations are within the scope that may be claimed.

The above-described hardware description is a non-limiting example of corresponding structure for performing the functionality described herein.

Obviously, numerous modifications and variations of the present disclosure are possible in light of the above teachings. It is therefore to be understood that within the scope of the appended claims, the invention may be practiced otherwise than as specifically described herein.

1. A smart watch for tremor monitoring and control of tremors in an extremity of a user, comprising:
a wearable band including:
a camera;
a plurality of motors;
an onboard communications module configured to communicate with a cloud server storing a tremor control application; and
a computing system including circuitry and a processor having program instructions configured to actuate the camera to take a series of images, generate an analysis of the series of images and actuate the motors to generate tremor control vibrations based on the analysis of the series of images.

2. The smart watch of claim 1, wherein the computing system is operatively connected to:
the camera;
the plurality of motors;
the onboard communications module;
wherein the computing system is further configured to:
monitor a tremor reduction effect of the vibrations;
generate data packets including the analysis of the series of images and the tremor reduction effect; and
wherein the onboard communications module is configured to transmit the data packets to the tremor control application.

3. The smart watch of claim 2, wherein the wearable band further comprises at least one of a plurality of sensors operatively connected to the computing system, the plurality of sensors including:
a body temperature sensor,
an O₂ pulse oximeter,
a heart rate monitor;
wherein the computing system is configured to receive at least one of a temperature, O₂ and heart rate measurement from the plurality of sensors and include the measurement in the data packets sent to the tremor control application.

4. The smart watch of claim 2, wherein the plurality of motors includes a set of motors which vibrate without emitting audible sound and a set of motors which vibrate and emit audible frequencies.

5. The smart watch of claim 3,
wherein the computing system further includes a memory storing log in credentials;
wherein the computing system is further configured to include the log in credentials in the data packet sent to the tremor control application.

6. The smart watch claim 5, wherein the tremor control application includes:
a transceiver configured to receive the data packets;
a registration module configured to register a computing device of the smart watch user with the tremor control application and generate a unique access code;
an application memory configured to store unique access codes of a plurality of smart watch users registered with the tremor control application;
a database configured to store a body profile of each smart watch user;
application circuitry and an application processor having program instructions configured to:
match the log in credentials to one of the unique access codes;
retrieve the body profile of the user associated with the unique access code;
update the database with the at least one sensor measurement, the analysis of the series of images and the tremor reduction effect;
compare the at least one sensor measurement, the analysis of the series of images and the tremor reduction effect to the body profile;
identify the onset of tremors;
detect magnitudes and frequencies of the tremors;
generate motor tuning parameters to reduce the tremors;
send the motor tuning parameters to the transceiver;
wherein the transceiver is further configured to transmit the motor tuning parameters to the onboard communications module of the smart watch.

7. The smart watch of claim 6, wherein the tremor control application further comprises:
a health report module configured to receive the at least one sensor measurement, the body profile and the tremor reduction effect and generate a health report;
wherein the transceiver is further configured to transmit the health report to at least one of the onboard communications module and the computing device of the smart watch user.

8. The smart watch of claim 6, wherein the motor tuning parameters include an amplitude and frequency range for each motor of the smart watch and optionally one or more of a pattern of motor actuation, a period of operation, time of day for monitoring tremors and a schedule for monitoring tremors.

9. The smart watch of claim 6,
wherein the onboard communications module is further configured to receive the motor tuning parameters;
wherein the computing system further includes a pulse width modulator configured to generate commands for each motor of the plurality of motors;
wherein the circuitry is configured to use the commands to actuate each motor according to the motor tuning parameters.

10. A method for monitoring and controlling tremors in an extremity of a patient by a smart watch, comprising:
actuating a camera located on the extremity to acquire a first series of images of an environment surrounding the extremity;
analyzing the first series of images to determine a magnitude and frequency of a tremor;
actuating at least one of a plurality of motors located on the extremity to generate tremor control vibrations based on the analysis of the first series of images.

11. The method of claim 10, wherein analyzing the first series of images comprises:
timestamping the images;
subtracting each image from an image having an immediately previous timestamp to generate a time series of differences;
normalizing the differences;
comparing a magnitude of each difference to a threshold;
identifying a tremor occurrence if the magnitude is greater than the threshold; and
determining a frequency of the tremors by mapping the tremor occurrences with respect to the timestamps.

12. The method of claim 11, further comprising;
retrieving log in credentials from a memory of the smart watch;
transmitting the log in credentials, magnitudes and frequencies in a data packet to a tremor control application;
receiving the data packet by the tremor control application;
matching the log in credentials to a database of access codes of the tremor control application;
retrieving a body profile of the patient from the database;
generating, by the tremor control application, motor tuning parameters based on the magnitudes, frequencies and the body profile;
transmitting the motor tuning parameters to the smart watch;
receiving, by the smart watch, the motor tuning parameters; and
generating commands by pulse width modulation to actuate each motor according to the motor tuning parameters.

13. The method of claim 11, further comprising:
registering the smart watch with the tremor control application by:
creating public and private pair keys by the smart watch;
including the public key in the data packet with the log in credentials;
transmitting the data packet to the tremor control application;
storing the public key as an access code in the database.

14. The method of claim 13, further comprising:
registering a computing device of the patient with the tremor control application by:
transmitting the public key to the tremor control application by the computing device;
matching the public key to the access code;
creating a body profile including at least one of age, height, previous tremor occurrence magnitudes and frequencies, gender, ethnic group, address, credit card information and medical diseases of the patient; and
generating a contact list including at least one of a preferred medical practitioner, a next of kin, a hospital, a medical alert center and a contact person.

15. The method of claim 14, further comprising:
monitoring a first tremor reduction effect due to the tremor control vibrations generated by a first motor and a second motor;
comparing the first tremor reduction effect to a threshold;
stopping the first and second motors if the first tremor reduction effect is 100%;
continuing to actuate the first and second motors if the first tremor reduction effect is greater than 50% and less than 100%;
actuating a third motor located between the first and second motor if the first tremor reduction effect is less than or equal to 50%;
monitoring a second tremor reduction effect due to the tremor control vibrations generated by the first, second and third motors;
comparing the second tremor reduction effect to the threshold;
stopping the first, second and third motors if the second tremor reduction effect is 100%;
continuing to actuate the first, second and third motors if the second tremor reduction effect is greater than 50% and less than 100%;
actuating fourth and fifth motors located opposite the first and second motors respectively to generate vibrations and audible frequencies if the second tremor reduction effect is less than or equal to 50%;
monitoring a third tremor reduction effect due to the tremor control vibrations generated by the first, second, third, fourth and fifth motors and the audible frequencies generated by the fourth and fifth motors;
comparing the third tremor reduction effect to the threshold;
stopping the first, second, third, fourth and fifth motors if the tremor reduction effect is 100%;
continuing to actuate the first, second, third, fourth and fifth motors if the tremor reduction effect is greater than 50% and less than 100%;
sending an alert to a contact person on the contact list if the third tremor reduction effect is less than or equal to 50%;
transmitting the first, second and third tremor reduction effects to the tremor control application; and
storing the first, second and third tremor reduction effects in the database with the body profile.

16. The method of claim 15, further comprising:
receiving, by a health report module of the tremor control application, a sensor measurement from at least one of a plurality of sensors located on the smart watch, wherein the plurality of sensors includes a body temperature sensor, an O₂ pulse oximeter and a heart rate monitor;
receiving, by the health report module, the body profile and first, second and third tremor reduction effects;
correlating the at least one sensor measurement, the body profile and first, second and third tremor reduction effects;
generating a health report; and
transmitting the health report to at least one of the onboard communications module and the computing device of the patient.

17. A smart watch system for monitoring and controlling tremors in an extremity of a patient, comprising:
a wearable band surrounding the extremity, the wearable band including:
a camera configured to acquire a first series of images of an environment surrounding the extremity;
an image processor configured to receive the first set of images and timestamp each image;
an image comparator configured to subtract each image from an image having an immediately previous timestamp to generate a time series of differences;
a tremor analysis module configured normalize the differences, compare the magnitude of each difference to a threshold, identify a tremor occurrence if the magnitude is greater than the threshold and determine a frequency of the tremors by mapping the tremor occurrences with respect to the timestamps;
a plurality of sensors including at least one of a body temperature sensor, an O2 pulse oximeter and a heart rate monitor the plurality of sensors configured to take measurements of the extremity;
a plurality of motors configured to generate at least one of vibrations in the extremity and vibrations in the extremity which include audible frequencies;
a pulse width modulator configured to generate commands to actuate the motors to generate the vibrations;
a memory storing log in credentials;
a computing system including circuitry and a processor having program instructions configured to:
actuate the camera to acquire the first series of images,
instruct the tremor analysis module to determine the frequency and magnitude of the tremors,
actuate at least one of the plurality motors to generate tremor control vibrations based on the analysis of the series of images;
monitor a first tremor reduction effect due to the vibration of the at least one motor;
generate a data packet including the at least one sensor measurement, the magnitude and frequency of the tremor occurrence, the first tremor reduction effect and the log in credentials;
an onboard communications module configured to transmit the data packet to a cloud server;
a tremor control application stored on the cloud server, the tremor control application including:
a transceiver configured to receive the data packets;
a registration module configured to register a computing device of the patient with the tremor control application and to generate a unique access code;
an application memory configured to store unique access codes of a plurality of smart watch patients registered with the tremor control application;
a database configured to store a body profile of the patient;
application circuitry and an application processor having program instructions configured to:
match the log in credentials to one of the unique access codes;
retrieve the body profile of the user associated with the unique access code;
update the database the at least one sensor measurement, the magnitude and frequency of the tremor occurrence, the first tremor reduction effect;
compare the at least one sensor measurement, the magnitude and frequency of the tremor occurrence, the first tremor reduction effect to the body profile;
generate motor tuning parameters to reduce the tremors, wherein the motor tuning parameters include an amplitude and frequency range for each motor of the smart watch;
send the motor tuning parameters to the transceiver; and
wherein the transceiver is further configured to transmit the motor tuning parameters to the onboard communications module of the smart watch.

18. The smart watch system of claim 17, the tremor analysis application further comprising:
a health report module configured to receive the at least one sensor measurement, the body profile and the tremor reduction effect and generate a health report;
wherein the transceiver is further configured to transmit the health report to at least one of the onboard communications module and the computing device of the patient.

19. The smart watch system of claim 17, wherein the computing system is further configured to monitor an effectiveness of the vibrations, by:
monitoring a first tremor reduction effect due to the tremor control vibrations generated by a first motor and a second motor;
comparing the first tremor reduction effect to a threshold;
stopping the first and second motors if the first tremor reduction effect is 100%;
continuing to actuate the first and second motors if the first tremor reduction effect is greater than 50% and less than 100%;
actuating a third motor located between the first and second motor if the first tremor reduction effect is less than or equal to 50%;
monitoring a second tremor reduction effect due to the tremor control vibrations generated by the first, second and third motors;
comparing the second tremor reduction effect to the threshold;
stopping the first, second and third motors if the second tremor reduction effect is 100%;
continuing to actuate the first, second and third motors if the second tremor reduction effect is greater than 50% and less than 100%;
actuating fourth and fifth motors located opposite the first and second motors respectively to generate vibrations and audible frequencies if the second tremor reduction effect is less than or equal to 50%;
monitoring a third tremor reduction effect due to the tremor control vibrations generated by the first, second, third, fourth and fifth motors and the audible frequencies generated by the fourth and fifth motors;
comparing the third tremor reduction effect to the threshold;
stopping the first, second, third, fourth and fifth motors if the tremor reduction effect is 100%;
continuing to actuate the first, second, third, fourth and fifth motors if the tremor reduction effect is greater than 50% and less than 100%;
sending an alert to a contact person on the contact list if the third tremor reduction effect is less than or equal to 50%;
transmitting the first, second and third tremor reduction effects to the tremor control application; and
storing the first, second and third tremor reduction effects in the database with the body profile.

20. The system of claim 19, wherein the plurality of motors equals five motors and the pulse width monitor is configured to generate commands to the plurality of motors in the range of 0-100% power with a standard deviation of 10%, wherein the command to:
a first motor is a randomly generated number of mean of 75%;
a second motor is a randomly generated number of mean of 65%;
a third motor is a randomly generated number of mean of 55%;
a fourth motor is a randomly generated number of mean of 45%; and
a fifth motor is a randomly generated number of mean of 35%.
